(19) United States
(12) Patent Application Publication
(10) Pub. No.: US 2026/0034324 A1
(43) Pub. Date: Feb. 5, 2026

(54) TRACHEOSTOMY TUBE WITH DISLODGEMENT ALARM

(71) Applicant: Christiana Care Health System, Inc., Wilmington, DE (US)

(72) Inventors: Leasha Roy, New Castle, DE (US); Emily Colonna, Wilmington, DE (US)

(21) Appl. No.: 19/286,528

(22) Filed: Jul. 31, 2025

Related U.S. Application Data
(60) Provisional application No. 63/677,915, filed on Jul. 31, 2024.

(51) Int. Cl. A61M 16/04; A61M 16/00

(52) U.S. Cl. CPC A61M 16/0465; A61M 16/0051; A61M 16/022; A61M 16/0497; A61M 2205/15; A61M 2205/3306; A61M 2205/3553; A61M 2205/581; A61M 2205/583; A61M 2230/06; A61M 2230/205

(57) ABSTRACT

A tracheostomy tube includes a dislodgement sensor-based system operable to detect partial and/or full-decannulation and resulting disruption to a person's airway. The tube's system provides an alert to a caregiver so that the caregiver can respond immediately, contemporaneously with a dislodgement event, and act to restore the person's airway, and avoid asphyxiation. The system includes a sensor operable to sense a vital sign of a person or a separation of the neck plate from a position abutting the person's neck, to detect a dislodgement state. The system may include a controller operatively connected to the sensor. The controller is configured to detect the dislodgement state as a function of data received from the sensor. In response to a dislodgement event, the caregiver may be alerted by an alarm device of the sensor-based system, or by another device for alerting the caregiver that is signaled by the tube's sensor-based system.

### CROSS-REFERENCE TO RELATED APPLICATION

This application claims the benefit under 35 U.S.C. § 119 (e) of U.S. Provisional Patent Application No. 63/677,915, filed Jul. 31, 2024, the entire disclosure of which is hereby incorporated by reference.

### FIELD OF THE INVENTION

The present invention relates generally to tracheotomy procedures and tracheostomy breathing tubes, and more particularly, to a tracheostomy tube including a dislodgement alarm configured to provide a notification to clinical care personnel in the event of accidental or unauthorized partial or complete decannulation resulting in a loss of a person's airway as the result of dislodgement and/or removal of the tracheostomy tube from the person's trachea.

### DISCUSSION OF RELATED ART

People may experience a need for breathing assistance as a result of an accident or a health/medical issue. Mechanical ventilator machines are configured to provide a supply of air, via a ventilator tube, to provide such breathing assistance.

In certain circumstances, a tracheotomy procedure is performed to provide the person with a tracheostomy tube. As well known in the art, the tracheotomy procedure involves creating a hole in the person's neck, through the person's skin and bodily tissues, so that the tracheostomy tube can be inserted through the hole and into the person's trachea/windpipe. When in use with a ventilator, an outer portion of the tracheostomy tube is connected to the ventilator tube of the ventilator machine, and an inner portion of the tracheostomy tube resides within the person's trachea to maintain an open airway and deliver the air supplied from the ventilator machine to the person's lungs via the tracheostomy tube and trachea, as well known in the art. As patients become more stable, patients will be weaned from ventilator support to only supplemental oxygen and humidification, and in this case, the ventilator tube is disconnected and the outer portion of the tracheostomy tube is not connected to anything. In a typical use case, the tracheostomy tube is essential or critical to maintaining the patient's airway and/or proper ventilation.

Disruption, partial dislodgement and/or complete removal (i.e., decannulation), referred to collectively herein as “dislodgement”, of the tracheostomy tube, whether deliberately or accidentally, can be a life-threatening event. Unauthorized/unsupervised self-decannulation can be a particular concern for persons with cognitive impairment. Unauthorized/unsupervised dislodgement generally requires an immediate response and intervention from the healthcare team to restore cannulation and/or otherwise restore the patient's airway and/or proper ventilation to prevent death by asphyxiation, etc.

Presently, dislodgement is generally identified during routine rounds by the care team (which may be asynchronous with and long after a dislodgement event), or by reporting of the event by a patient's visitor, or if the patient is being remotely monitored, e.g., by changes in blood oxygen saturation and/or heart rate as detected by purpose-specific sensors and associated monitoring equipment, which can only indirectly suggest that there might be a tracheostomy dislodgement issue. Further, abnormal blood oxygen and/or heart rate do not actually/directly detect decannulation/dislodgement, but rather detects results that may occur asynchronous after a time period after such dislodgement, and thus cannot serve to ensure swift rectification of any breathing issue in the event of dislodgement. Accordingly, though maintenance of the airway is critical to patient health, detection of a tracheostomy tube dislodgement may be delayed relative to occurrence of the dislodgement, which jeopardies the patient's health, and is therefore undesirable.

What is needed is an improved approach for detection of dislodgement of a tracheostomy tube that allows for swift detection of a dislodgement that could jeopardize the patient's airway and health, and thus allows swift rectification of any breathing issue to ensure that adequate patient ventilation is maintained.

### SUMMARY

The present invention provides a tracheostomy tube with a dislodgement sensor-based system that provides an improved approach for detection of dislodgement of a tracheostomy tube and allows for swift detection of a dislodgement that could jeopardize the patient's airway and health. The tracheostomy tube includes generally-conventional tracheostomy tube structures, and a dislodgement sensor-based system configured to detect a dislodgement state in which the tracheostomy tube has become dislodged (partially or fully) from its intended location supporting the person's airway.

Accordingly, an exemplary tracheostomy tube comprises generally conventional structures such as a neck plate; a first sidewall supported on said neck plate and defining an inner cannula configured to admit passage of a flow of air; a second sidewall supported on said neck plate and defining an outer cannula in fluid communication with an inflatable cuff driven by a pilot balloon. Additionally, an exemplary tracheostomy tube comprises: a dislodgement sensor-based system comprising: a sensor operable to sense one of a vital sign of a person and a disengagement of the neck plate from a position abutting skin of a neck of the person; and a controller operatively connected to the sensor, the controller being operable to detect a dislodgement state as a function of data received from said dislodgement sensor.

The tracheostomy tube may also comprise an alarm device operable to provide an alarm signal. In such an embodiment, the controller is configured to issue a control signal to control the alarm device to issue the alarm signal when the controller detects the dislodgement state.

The tracheostomy tube may also comprise a data communication module operatively connected to the controller and operable to transmit a control signal to a remote device to cause the remote device to provide an alert signal when the controller detects the dislodgement state.

Accordingly, the tracheostomy tube with a dislodgement sensor-based system is configured to detect a dislodgement state (including dislodgement and/or circumstances suggesting dislodgement is imminent) and to responsively alert a caregiver/hospital staff to the dislodgement state in a prompt manner, responsive to the dislodgement, so that the caregiver can start interventions synchronously with (or very soon after) any dislodgement event, or problematic changes in vital signs that are associated with a dislodgement event, to prevent an airway code or cardiac arrest. Additionally, the tracheostomy tube with dislodgement sensor can help to avoid/prevent dislodgement by allowing staff to intervene when a dislodgement appears imminent, before any problematic change in vital signs.

### BRIEF DESCRIPTION OF THE FIGURES

An understanding of the following description will be facilitated by reference to the attached drawings, in which:

FIG. 1 is a side view of a person in a hospital bed connected to a mechanical ventilator machine to receive breathing assistance/air via an exemplary tracheostomy tube having a dislodgement sensor-based system in accordance with the present invention; and

FIGS. 2 and 3 are perspective views of an exemplary tracheostomy tubes of the prior art;

FIGS. 4 and 5 are perspective views of an exemplary tracheostomy tube with a dislodgement-based sensor system in accordance with exemplary embodiments of the present invention; and

FIG. 6 is a schematic block diagram of an exemplary tracheostomy tube with dislodgement sensor-based system in accordance with exemplary embodiments of the present invention.

### DETAILED DESCRIPTION

The present invention provides a tracheostomy tube having generally-conventional tracheostomy tube structures, and also including a dislodgement sensor-based system configured to detect a dislodgement state in which the tracheostomy tube has become dislodged (partially or fully), in accordance with the present invention.

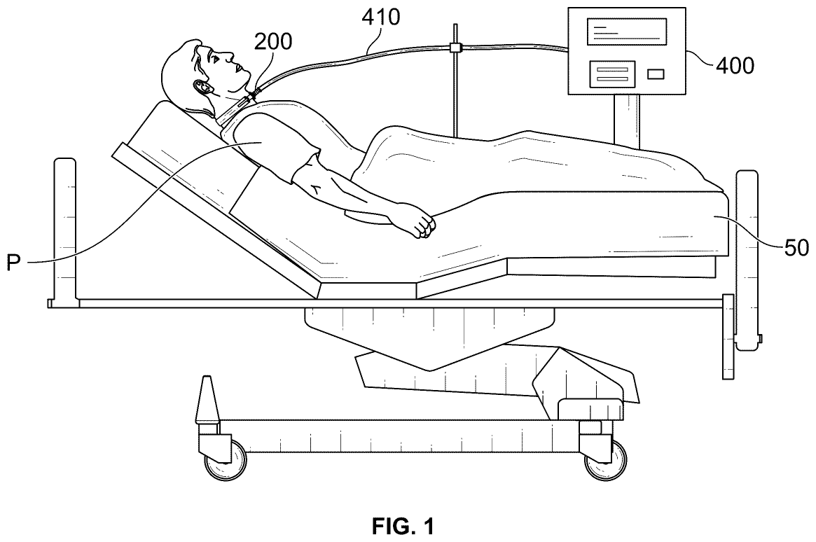

FIG. 1 is a side view of a person P lying in a hospital bed 50. The person P has an exemplary tracheostomy tube 200 (having a dislodgement sensor-based system in accordance with the present invention) secured to the person's neck. In this example, the tracheostomy tube 200 is connected to a mechanical ventilator machine 400 to receive breathing assistance/air via the tracheostomy tube.

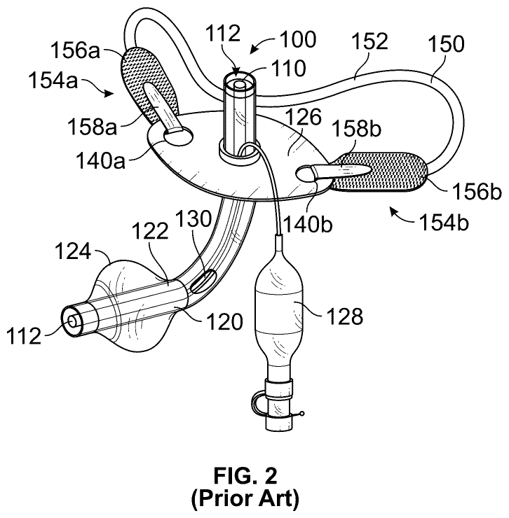

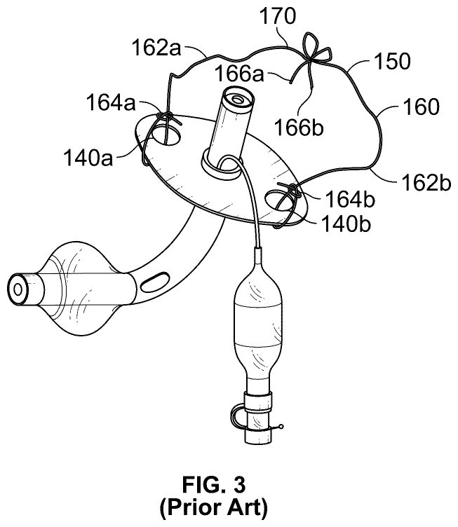

A tracheostomy tube 200 having a dislodgement sensor-based system 300 in accordance with the present invention may be similar in structure to conventional tracheostomy tubes. As best shown in FIGS. 2 and 3, a conventional tracheostomy tube 100 of the prior art may include a first sidewall 110 defining an inner cannula 112 for admitting passage of a flow of air from a ventilator machine 400 via a ventilator supply tube 410. Additionally, the tracheostomy tube 200 may include a second sidewall 120 defining an outer cannula 122 that flows air to/from an inflatable/deflatable cuff 124 as driven by a pilot balloon 128, as known in the art. In this example, the exemplary tube 100 is fenestrated, meaning that the first and second sidewalls 110, 120 define an opening 130, though fenestration is not required. The sidewalls 110, 120 and cuff 124 are supported on a flange/neck plate/faceplate 126.

To maintain the tracheostomy tube 100 in an operative position in which the tube has been properly inserted into the person's trachea/windpipe, the tube is typically secured to the person's body/neck by a strap. In one common example, a neck plate 126 of the tracheostomy tube 100 defines a pair of openings 140a, 140b through which a strap 150 is passed for securing purposes. In one exemplary known embodiment, the strap 150 has a neck band body 152 that is soft or padded for patient comfort when applied against the skin, and a pair of ends 154a, 154b that are passed through the neck plate 126. Further, each end 154a, 154b has complementary fields of hook fastener and loop fastener of a hook-and-loop fasteners system, such as that sold commercially as Velcro® fastener. For example, each end 154a, 154b. may include a landing field 156a, 156b of hook fastener and a distal tail field 158a, 158b of loop fastener that can be passed through an opening 140a, 140b, and be pressed against, and thereby mated to, a respective landing field 156a, 156b to secure the strap 150 (and thereby the tracheostomy tube 100) around the person's neck, with the neck plate 126 abutting the skin of the person's neck and the cuff 124 and a portion of the tracheostomy tube 100 positioned in the person's P esophagus, as will be appreciated from FIGS. 1 and 2.

Referring now to FIG. 3, in another common example, the strap 150 is provided with a neck band body 160 in the nature of a tie that includes a first portion 162 having a first end 164a secured to the neck plate 126 through the first opening 140a and a second end 166a opposite the first end, and a second portion 162b having a second end 164b secured to the neck plate 126 through the second opening 140b and a second end 166b opposite the first end, such that the second ends 166a, 166b are free ends that can be looped around the person's neck and be tied at the back of the next in a loose knot 170 to secure the strap 150 (and thereby the tracheostomy tube 100) around the person's neck, with the neck plate 126 abutting the skin of the person's neck and the cuff 124 and a portion of the tracheostomy tube 100 positioned in the person's P esophagus, as will be appreciated from FIG. 3.

However, these straps are easily unfastened, and are not designed to prevent someone from loosening them and decannulating. More particularly, the knot of the tie is easy to locate and can be easily undone by the patient, and similarly, the free ends of the hook-and-loop embodiment are typically located at the front of the neck where they may be easily located and unsecured, either accidentally or intentionally by the patient (who is unauthorized to perform decannulation).

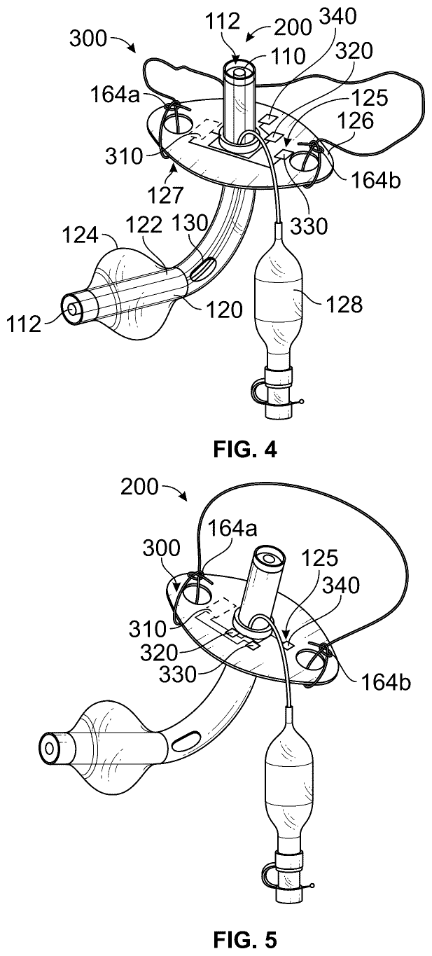

Accordingly, in addition to the structures of a conventional tracheostomy tubes, a tracheostomy tube 200 of the present invention includes not only conventional tracheostomy structures, but also a dislodgement sensor-based system 300, as shown in FIGS. 4 and 5. The sensor is operator to detect a decannulation or other dislodgement of the tracheostomy tube immediately upon dislodgement, and to responsively cause an alert to be issued (e.g., sounding of an alarm, transmitting of an alarm or control signal, message, etc.) that allows for swift detection of the dislodgement, to facilitate swift rectification of any breathing issue, and to ensure that adequate patient ventilation is maintained to avoid jeopardizing the patient's airway and health, and a resulting monetary loss to a hospital, caregiver, etc.

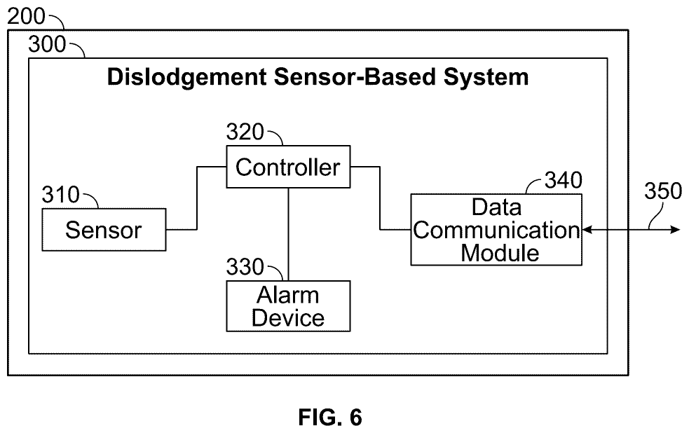

FIGS. 4 and 5 are perspective views of an exemplary tracheostomy tube 200 with a dislodgement sensor-based system 300, comprising at least one sensor, in accordance with exemplary embodiments of the present invention. FIG. 6 is a schematic block diagram of the exemplary tracheostomy tube 200 of FIGS. 4 and 5. Referring now to FIGS. 4-6, in the exemplary embodiment of FIGS. 4 and 5, the dislodgement sensor-based system 300 includes a sensor 310 disposed on an inside/underside 127 of the neck plate 126, so that it is positioned to touch the skin of a person's neck when the tracheostomy tube 200 is in use and secured to the patient's neck in an operable position for delivering a flow of air to the patient via the tracheostomy tube 200 (e.g., as shown in FIG. 1).

In one exemplary embodiment, the sensor 310 is configured to measure at least one of a blood oxygen level and a heart rate percutaneously, while abutting/in contact the skin. By way of example, a commercially-available pulse oximeter sensor (SpO₂ sensor) (e.g., including one or more LEDs and a photodetector), near-infrared spectroscopy (NIRS) sensor (using an array of near-infrared light sources to assess tissue oxygenation), or photoplethysmography (PPG) sensor may be used to measure/gather data associated with the person's blood oxygen level percutaneously. By way of further example, a commercially-available photoplethysmography (PPG) sensor, electrocardiography (ECG), ballistocardiography (BCG), seismocardiography (SCG) or acoustic/phonocardiography sensor may be used to measure/gather data associated with the person's heart rate percutaneously. Accordingly, a suitable sensor may gather blood oxygen level and/or heart rate data operating according to optical, electrical, mechanical, and/or acoustic measurement principles. Any suitable sensor may be used to gather blood oxygen level or heart rate data, or other vital sign-type data, as will be appreciated by those skilled in the art.

In another exemplary embodiment, the sensor 310 alternatively comprises a mechanical switch or sensor operable to sense a separation of the neck plate 126 from the skin of the person's neck. By way of example, a mechanical limit switch, a tactile/push-button/toggle switch, a leaf switch, a snap-action or microswitch, or a pressure sensor (such as a force sensing resistor) may be used for this purpose. Depending on the type of sensor used, the sensor 310 may be mounted on the interior/underside 127 or outside/topside 125 of the neck plate 126, according to the mechanism of operation of the sensor, as will be appreciated by those skilled in the art.

In yet another exemplary embodiment, the sensor 310 alternatively comprises a photosensor or other optical sensor operable to sense a separation of the neck plate 126 from the skin of the person's neck. Depending on the type of sensor used, the sensor 310 may be mounted on the interior/underside 127 or outside/topside 125 of the neck plate 126, according to the mechanism of operation of the sensor, as will be appreciated by those skilled in the art.

Accordingly, a suitable sensor may gather blood oxygen level and/or heart rate data, or faceplate position/skin contact data/signals operating according to optical, electrical, magnetic, mechanical, and/or acoustic measurement principles. Any suitable sensor may be used to gather blood oxygen level or heart rate data, or other vital sign-type data, as will be appreciated by those skilled in the art.

In accordance with the present invention, the sensor-based system 300 further includes a controller 320, which is operatively connected to the sensor 310 for data communication therewith. The controller 320 is configured to receive and/or process data/a signal received from the sensor and determine whether data from the sensor 310 indicates that a measured value (e.g., blood oxygen level or heart rate) is undetectable or below an acceptable threshold (e.g., in the case of blood oxygen level or heart rate) or to sense physical separation/demounting of the neck plate 126 of the tracheostomy tube 200 from the person's neck/skin. If the controller determines that a measured value is undetectable or below an acceptable threshold, or that the neck plate 126 has been separated from contact with the skin of the neck, then dislodgement of the tracheostomy tube 200 is presumed, and the controller 320 operates to take action to initiate an alert signal/warning/alarm for warning a caregiver, etc. that the tracheostomy tube 200 is dislodged. More particularly, the controller 320 operates to cause an alert signal/warning/alarm to be issued from the controller 320/tracheostomy tube 200, so that a caregiver is alerted to check on the person and take action as needed to restore the person's airway, etc. The controller 320 may be supported anywhere on the tracheostomy tube, e.g., on the outside/topside 125 of the neck plate 126.

In certain embodiments, as in the example of FIGS. 4-6, the dislodgement sensor-based system 300 may further include an alarm device 330 providing an alarm signal for alerting a caregiver, and the controller 320 may include suitable circuitry, hardware, software or other logic or mechanisms to cause the controller 320 to detect a dislodgement state by determining if/when the dislodgement sensor has detected a dislodgement of the tracheostomy tube or other circumstance suggesting dislodgement is imminent, and to issue a control signal to control the alarm device 330 to cause the alarm device 330 to generate/issue the alarm signal when such a dislodgement state is detected. The alarm device 330 may be any sort of device for issuing an audible or visible alarm signal perceptible to a human caregiver. By way of example, the alarm device 330 may include a piezoelectric or electromagnetic buzzer, a speaker, or a sounder for issuing an audible signal, or an LED or light bulb for issuing a visible signal. Accordingly, the tracheostomy tube 200 may be configured to issue an alarm signal to directly alert a caregiver and cause the caregiver to check on the person and take action as needed to restore the person's airway, etc.

In certain embodiments, as in the example of FIGS. 4-6, the dislodgement sensor-based system 300 further includes a data communication module 340 operatively coupled to the controller 320, and the controller 320 may include suitable circuitry, hardware, software or other logic or mechanisms to cause the controller 320 to detect a dislodgement state by determining if/when the dislodgement sensor has detected a dislodgement of the tracheostomy tube or other circumstance suggesting dislodgement is imminent, and to issue a first control signal to control the data communication module 340 to cause the data communication module 340 to transmit data and/or a control data signal (collectively, “second control signal”) to a remotely-located or otherwise separate device to cause that separate device to issue an alert signal for alerting a caregiver. By way of example, the separate device may be any sort of device and may include an alarm device for issuing an audible or visible alarm signal perceptible to a human caregiver. By way of example, the separate device may include a piezoelectric or electromagnetic buzzer, a speaker, or a sounder for issuing an audible signal, or an LED or light bulb for issuing a visible signal. By way of alternative example, the separate device may be configured to display a message, such as text or graphics displayed on a display screen of the separate device. In either case, the data communication module 340 transmits to a separate device a control signal causing the separate device to alert a clinician or other member of the person's care team so that the removal of the tracheostomy tube can be avoided, or correct placement of the tracheostomy tube can be restored, or so the person can otherwise be provided by adequate ventilation. By way of example, the data communication module may include a data transmitter or transceiver capable of transmitting/communicating data via a data communication channel 350 to another device, e.g., via wired or wireless data transmission. Accordingly, the tracheostomy tube 200 may be configured to issue a control signal directly to another device that can issue an alert signal to directly alert a caregiver and cause the caregiver to check on the person and take action as needed to restore the person's airway, etc.

A battery may be supported on the neck plate126 or elsewhere on the tracheostomy tube 200 to power the components of the dislodgement sensor-based system 300.

In certain embodiments, the sensor may be operatively connected directly to the alarm device and the controller may be omitted. In such a configuration, the alarm device is operable to issue the alarm signal as a function of the signal received from the sensor.

In use, a tracheostomy tube 200 with a dislodgement sensor-based system 300 in accordance with the present invention may be inserted into a person's trachea, and be secured to a person's neck, using conventional site preparation and insertion techniques and procedures. As is conventional, this involves securing the tracheostomy tube 200 to a person's neck, with the neck plate 126 abutting the skin of the person's neck.

After the tracheostomy tube 200 has been secured to the person's neck in the usual manner, the sensor 310 of the dislodgement sensor-based system 300 is generally abutting the skin of the person's neck. In this position, depending upon the particular type of sensor 310 employed, is in a position to sense a dislodgement state (which may involve disturbance, partial decannulation or full decannulation). Dislodgement may be due to bodily movements of the person, and may be accidental or intentional in nature.

A data signal or other signal from the sensor(s) 310 is received by the controller 320, which may process, interpret, or otherwise act on the data/signal. For example, a sensor 310 capable of detecting blood oxygen level or heart rate will detect a decrease or irregularity or discontinuation of receipt of associated data, which indicates a dislodgement state. By way of alternative example, a sensor 310 capable of detecting physical engagement with/abutting with the skin of the neck will detect a removal or displacement of the neck plate 126 from the skin, which indicates a dislodgement state. By way of another alternative example, a sensor 310 capable of detecting physical engagement with/abutting with the skin of the neck will detect a removal or displacement of the neck plate 126 from the skin, which indicates a dislodgement state.

If the controller 320 determines that the data/signal from the sensor(s) 310 indicates a dislodgement state, then the controller 320 will initiate action to provide an alarm/alert to a caregiver, to prompt the caregiver to check on the person and remedy any dislodgement or otherwise restore the person's airway, etc.

For example, in embodiments in which the tracheostomy tube 200 and dislodgement sensor-based system 300 include an alarm device, the controller 320 may issue a control signal to the alarm device 330 to control the alarm device 330 to cause the alarm device 330 of the tracheostomy tube 200 to generate/issue an audible or visual alarm signal when such a dislodgement state is detected.

By way of further example, in embodiments in which the tracheostomy tube 200 and dislodgement sensor-based system 300 include a data communication module 340, the controller 320 may issue a first control signal to control the data communication module 340 to cause the data communication module 340 to transmit data and/or a control data signal (collectively, “second control signal”) to a remotely-located or otherwise separate device, to cause that separate device to issue an alert signal for alerting a caregiver.

Accordingly, in the event of detection of a dislodgement state, the dislodgement sensor-based system 300 and tracheostomy tube 200 act to directly or indirectly alert a caregiver, so that the caregiver may take action to ensure that the patient's airway is maintained.

Accordingly, the present invention provides a tracheostomy tube with a dislodgement sensor-based system that is configured to detect a dislodgement state (including dislodgement and/or circumstances suggesting dislodgement is imminent) and to responsively alert a caregiver/hospital staff to the dislodgement state in a prompt manner, responsive to the dislodgement, so the caregiver can start interventions synchronously with (or soon after) any dislodgement event, or problematic changes in vital signs that are associated with a dislodgement event, to prevent an airway code or cardiac arrest. Additionally, the tracheostomy tube with dislodgement sensor can help to avoid/prevent dislodgement by allowing staff to intervene when a dislodgement appears imminent, before any problematic change in vital signs.

Although the present invention is described above primarily in relation to a hospital setting, it should be noted that the device of the present invention may also be used in long-term care and skilled nursing facilities and in patients' homes, and may be particularly advantageous for use with pediatric patients.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A tracheostomy tube comprising:
a neck plate;
a first sidewall supported on said neck plate and defining an inner cannula configured to admit passage of a flow of air;
a second sidewall supported on said neck plate and defining an outer cannula in fluid communication with an inflatable cuff driven by a pilot balloon; and
a dislodgement sensor-based system comprising:
a sensor operable to sense one of a vital sign of a person and a disengagement of the neck plate from a position abutting skin of a neck of the person; and
a controller operatively connected to the sensor, the controller being operable to detect a dislodgement state as a function of data received from said sensor.

2. The tracheostomy tube of claim 1, wherein the sensor comprises a transcutaneous blood oxygen sensor.

3. The tracheostomy tube of claim 2, wherein the controller is configured to detect the dislodgement state when the transcutaneous blood oxygen sensor is unable to detect a blood oxygen level.

4. The tracheostomy tube of claim 2, wherein the controller is configured to detect the dislodgement state when data from the transcutaneous blood oxygen sensor indicates a blood oxygen level below an established threshold value.

5. The tracheostomy tube of claim 1, wherein the sensor comprises a transcutaneous heart rate sensor.

6. The tracheostomy tube of claim 5, wherein the controller is configured to detect the dislodgement state when the transcutaneous heart rate sensor is unable to detect a heart rate value.

7. The tracheostomy tube of claim 5, wherein the controller is configured to detect the dislodgement state when data from the transcutaneous heart rate sensor indicates a heart rate below an established threshold value.

8. The tracheostomy tube of claim 1, wherein the sensor is configured to sense a separation of the neck plate from skin of the person's neck.

9. The tracheostomy tube of claim 8, wherein the sensor comprises a mechanical switch operable to move when the neck plate is separated from skin of the person's neck.

10. The tracheostomy tube of claim 8, wherein the sensor comprises an optical sensor.

11. The tracheostomy tube of claim 10, wherein the sensor comprises a photosensor.

12. The tracheostomy tube of claim 1, further comprising an alarm device operable to provide an alarm signal, the controller being configured to issue a control signal to control the alarm device to issue the alarm signal when the controller detects the dislodgement state.

13. The tracheostomy tube of claim 12, wherein the alarm device is configured to issue an audible alarm signal.

14. The tracheostomy tube of claim 12, wherein the alarm device is configured to issue a visible alarm signal.

15. The tracheostomy tube of claim 1, further comprising a data communication module operatively connected to the controller and operable to transmit a control signal to a remote device to cause the remote device to provide an alert signal.

16. The tracheostomy tube of claim 1, further comprising a battery supported on the neck plate and operatively connected to at least one of the sensor and the controller.

17. The tracheostomy tube of claim 1, further comprising a strap securable to the neck plate to secure the tracheostomy tube around the person's neck.

18. A tracheostomy tube comprising:
a neck plate;
a first sidewall supported on said neck plate and defining an inner cannula configured to admit passage of a flow of air;
a second sidewall supported on said neck plate and defining an outer cannula in fluid communication with an inflatable cuff driven by a pilot balloon; and
a dislodgement sensor-based system comprising:
a sensor operable to sense one of a vital sign of a person and a disengagement of the neck plate from a position abutting skin of a neck of the person; and
an alarm device operatively connected to the sensor and operable to provide an alarm signal as a function of a signal received from the sensor.

19. The tracheostomy tube of claim 18, wherein the sensor comprises at least one of a transcutaneous blood oxygen sensor and a transcutaneous heart rate sensor.

20. The tracheostomy tube of claim 18, wherein the sensor comprises at least one of a mechanical switch and an optical sensor configured to sense a separation of the neck plate from skin of the person's neck.

21. The tracheostomy tube of claim 18, further comprising a controller operatively connected to the sensor and the alarm device, the controller being operable to detect a dislodgement state as a function of data received from said dislodgement sensor, and to issue a control signal to the alarm device to cause the alarm device to provide the alarm signal when the dislodgement state is detected.

22. A tracheostomy tube comprising:
a neck plate;
a first sidewall supported on said neck plate and defining an inner cannula configured to admit passage of a flow of air;
a second sidewall supported on said neck plate and defining an outer cannula in fluid communication with an inflatable cuff driven by a pilot balloon; and
a dislodgement sensor-based system comprising:
a sensor operable to sense one of a vital sign of a person and a disengagement of the neck plate from a position abutting skin of a neck of the person;
a controller operatively connected to the sensor, the controller being operable to detect a dislodgement state as a function of data received from said dislodgement sensor;
an alarm device operable to provide an alarm signal, the controller being configured to issue a control signal to control the alarm device to issue the alarm signal when the controller detects the dislodgement state; and
a data communication module operatively connected to the controller and operable to transmit a control signal to a remote device, when the controller detects the dislodgement state, to cause the remote device to provide an alert signal.

23. The tracheostomy tube of claim 22, wherein the sensor comprises at least one of a transcutaneous blood oxygen sensor and a transcutaneous heart rate sensor.

24. The tracheostomy tube of claim 22, wherein the sensor comprises at least one of a mechanical switch and an optical sensor configured to sense a separation of the neck plate from skin of the person's neck.
